(19) United States
(12) Patent Application Publication
(10) Pub. No.: US 2006/0217321 A1
(43) Pub. Date: Sep. 28, 2006

(54) Compositions to improve tic disorders

(75) Inventor: Makoto Ozeki, Yokkaichi (JP)

Correspondence Address: BURR & BROWN, PO BOX 7068, SYRACUSE, NY, 13261-7068, US

(73) Assignee: Taiyokagaku Co., Ltd., Yokkaichi (JP)

(21) Appl. No.: 11/387,448

(22) Filed: Mar. 23, 2006

(30) Foreign Application Priority Data
Mar. 24, 2005 (JP) 2005-086030

(51) Int. Cl. A61K 38/04; A61K 47/00; A61K 9/20

(52) U.S. Cl. 514/19; 424/439; 424/464

(57) ABSTRACT

Tic disorders can be improved by administering theanine, an amino acid that crosses the blood-brain barrier, or a dipeptide consisting of theanine and a different amino acid. In addition, these compositions can be added to foods and drinks or pharmaceuticals.

### TECHNICAL FIELD

The present invention relates to compositions to improve tic disorders, which contain an amino acid such as theanine and a dipeptide that cross the blood-brain barrier as an active ingredient. In addition, the present invention relates to the production methods for compositions to improve tic disorders.

### BACKGROUND ART

Tic disorders are defined by accidental, rapid, repetitive, non-rhythmic, and stereotyped movements or phonation, and by that these tic symptoms develop under 18 years of age and last for more than 4 weeks. In addition, tic disorders are classified into (1) transient tic disorder, (2) chronic motor or vocal tic disorder, and (3) Tourette's disorder. Transient tic disorder and chronic motor or vocal tic disorder are often present between late infancy and late childhood, whose incidence is reported to be 10-24% in all children with peak incidence around 7 years of age. The incidence is 1.5-3 times higher in boys than girls. Tourette's disorder occurs in 4-5 persons per 10,000, which account for 0.2-0.5% of all patients with tic disorders.

Tic disorders had long been considered to be caused by mental problems such as various stresses or latent conflict with domineering parents, but more recently they have been considered to be one of motor abnormalities that are seen during the development of the nervous system. In particular, the symptoms of Tourette's disorder develop age-dependently; thus, hereditary predisposition is presumed to underlie the onset.

To respond and treat tic disorders, the pathophysiology and natural course of tic disorders are first explained to the patient and patient's family to remove their anxiety. When children are forced to stop tics, they often become more nervous with resulting exacerbation; thus, consideration must be given not to force them. When there are evident psychological stresses at school and at home (these may have more or less exacerbated the tics at that time), the environments are controlled to remove them, but when various symptoms develop at 1 or more years after the onset, effects cannot usually be expected only with psychological care.

When there is no therapeutic effect by psychological care, drug therapy is conducted. Haloperidol, a major tranquilizer, is a commonly prescribed therapeutic agent for tic disorders. However, haloperidol does not cure tic disorders of all patients. In addition, the adverse effects of haloperidol, including drowsiness, fatigue, sedation, dizziness, vertigo, thirst, mydriasis, convergence insufficiency, increased appetite, weight gain, extrapyramidal dysfunction, mental depression, dysuria, liver damage, blood disorder, intestinal paralysis, and malignant syndrome, may develop.

Amino acids are constituents of proteins; thus, after being ingested in the diet, they are used as materials to synthesize various physiologically active substances, as well as in energy metabolism and as materials for body composition. Some amino acids cross the blood-brain barrier. The pathways, known to transport amino acids to the brain, include system A that transports short-chain neutral amino acids, mainly alanine, system L that transports long-chain neutral amino acids, represented by leucine, and system ASC that transports alanine, serine, and cysteine. Theanine has traditionally been known as tea extracts, and is the most abundant flavor ingredient among the amino acids contained in green tea; recently, theanine has been reported to have various effects, including control of the concentrations of physiologically active amines within the brain, stabilization of blood pressure, and sedation, and in addition, to cross the blood-brain barrier via the system L (Nonpatent Document 1).

Peptides are formed via bindings between multiple amino acids; thus, dipeptides are formed via a binding between 2 amino acids. Dipeptides have physiologic activities that a single amino acid does not exhibit. For instance, anticancer activity (Patent Document 1), anti-AIDS activity (Patent Documents 2 to 4), and pressor effects (Patent Document 5 and 6) are known.

[Patent Document 1] Japanese Translation of International Application (Kohyo) No. 2004-510831

[Patent Document 2] Japanese Published Unexamined Patent Application No. 2002-167397

[Patent Document 3] Patent No. 3408379

[Patent Document 4] International Publication WO01/047948

[Patent Document 5] Japanese Published Unexamined Patent Application No. 2003-48849

[Patent Document 6] Japanese Published Unexamined Patent Application No. 2004-099552

[Nonpatent Document 1] Yokogoshi, H., M. Kobayashi, et al., Effect of theanine, r-glutamylethylamide, on brain monoamines and striatal dopamine release in conscious rats, Neurochem Res, 23 (5) 667-673, 1998.

### DISCLOSURE OF INVENTION

### Problems to be Solved by the Invention

As described above, treatment methods to improve tic disorders include psychological care, although the treatment often extends over a long period of time. In addition, therapeutic agents for tic disorders often cause adverse effects.

The present invention was made in light of the above-mentioned circumstances; thus, the aim is to provide safe adverse-effect free compositions to improve tic disorders.

### MEANS FOR SOLVING THE PROBLEMS

As a result of a keen examination of safe adverse-effect free means to improve tic disorders, the inventors discovered improvement effects of theanine, a kind of amino acid that crosses the blood-brain barrier, and of dipeptides containing theanine, and have basically reached completion of the present invention. Theanine is an amino acid contained in tea, which has immediate effects besides being free of adverse effects. The above-mentioned effects of the compositions in the present invention are novel effects that the inventors first discovered.

Namely, the present invention includes the following (1)-(10):

(1) Compositions to improve tic disorders, which contain an amino acid that crosses the blood-brain barrier and/or a peptide with an amino acid that crosses the blood-brain barrier as an active ingredient.

(2) Compositions to improve tic disorders, wherein the amino acid according to (1) is theanine.

(3) Compositions to improve tic disorders, wherein the peptide according to claim 1 is a dipeptide consisting of (A) theanine and (B) one of the amino acids selected from theanine, glutamine, alanine, asparagine, asparatic acid, glutamic acid, glycine, histidine, isoleucine, leucine, lysine, methionine, phenylalanine, proline, serine, threonine, tryptophan, tyrosine, valine, arginine, and cysteine.

(4) Foods, drinks, and pharmaceuticals containing the composition described in (1).

(5) Foods, drinks, and pharmaceuticals containing the composition described in (2).

(6) Foods, drinks, and pharmaceuticals containing the composition described in (3).

(7) Production methods of tablets containing the composition described in (1).

(8) Production methods of tablets containing the composition described in (2).

(9) Production methods of tablets containing the composition described in (3).

(10) Production methods of tablets containing the composition described in (4).

### EFFECT OF THE INVENTION

According to this invention, safe adverse-effect free compositions to improve tic disorders can be provided.

### BEST MODE FOR CARRYING OUT THE INVENTION

Then, examples of the present invention are explained referring to the drawings, although the technical scope of the present invention is not limited to them; thus, the present invention can be implemented in various modes without changing the subject matter of the invention. In addition, the technical scope of the present invention covers an equivalent range.

The blood-brain barrier in the present invention is a structure that functions like a barrier between the blood and brain to prevent the unregulated passage of the blood substances into the brain. Some amino acids selectively cross the blood-brain barrier, and there are several kinds of transport systems. The amino acids used in the present invention are those that cross the blood-brain barrier, particularly those transported via system L. The amino acids transported via the system L are long-chain neutral amino acids, including phenylalanine, tyrosine, leucine, isoleucine, valine, histidine, methionine, threonine, and theanine.

The theanine used in the present invention is a glutamic acid derivative contained in tea leaves, a major flavor ingredient that is used as a food additive for flavoring. Production methods of the theanine, used in the present invention, include extraction from tea leaves, organic synthesis (Chem. Pharm. Bull., 19(7) 1301-1307 (1971)), reaction of the mixture of glutamine and ethylamine with glutaminase (Japanese Published Examined Patent Application No. H07-55154), increase of the accumulation of theanine in cultured cells while promoting the growth of cultured cells of tea in the medium containing ethylamine (Japanese Published Unexamined Patent Application No. H05-123166), and substitution of ethylamine for an ethylamine derivative such as ethylamine hydrochloride (Japanese Published Unexamined Patent Application No. 2000-026383), and any of these methods can be used. Here, the tea leaves include green tea, oolong tea, and black tea. L-, D-, and DL-theanine are available with these methods, of which L-theanine, approved as a food additive, can easily be used also from an economical viewpoint and thus is preferred in the present invention.

As for the theanine used in the present invention, purified product (theanine content: >98%), crude product (theanine content: 50-98%), and extracted essence (theanine content: 1-50%) serve the needs.

The theanine used in the present invention is sufficiently safe; for instance, neither death nor abnormality in general conditions and body weight was observed by the oral administration of 5 g/kg in an acute toxicity test using mice. In particular, theanine is known as a flavor ingredient of tea, and is used as a food additive for flavoring; thus, the Food Sanitation Law does not impose any quantity-limit on its use.

The tic of the tic disorders in the present invention is a disease with involuntary movements of specific muscles that are repeated frequently, and is characterized in being more common in later childhood, when the muscles and nervous system involved in physical movement develop and become more active, and thus is an important disease in pediatrics. There are symptoms at various stages, including mild cases only with blinking, which parents and people around the patient do not notice, and severe cases like Tourette's (Gilles de la Tourette) syndrome that interferes with a patient's daily life. Etiologies are classified into either symptomatic ones after encephalitis or essential ones; essential etiologies are classified into either organic ones with abnormalities in the cranial nerve or psychogenic ones, although the distinction is not clear. Movements themselves occur involuntarily regardless of the areas affected and severity of symptoms; thus, in terms of organic causes, tics are considered to be some disorders of the cranial nerve, centering on the extrapyramidal system, and are strongly influenced also by psychological factors.

Tics are sudden, rapid, repetitive, nonrhythmic, stereotyped, and localized involuntary movements of muscles, and include a motor tic and vocal tic. Tics occur involuntarily, but can be controlled voluntarily for a short period of time. However, rebound increase occurs subsequently. Tics disappear or decrease markedly during sleep. Motor tics appear most commonly in the face, followed by the head, neck, and shoulders, and are uncommon in the legs chest and abdomen. In those with vocal tics, tics are more common in sites other than the face. Tic disorders are classified in 307.20-307.23 of the Diagnostic and Statistical Manual of Mental Disorders—Fourth Edition (DSM-IV)” issued by the American Psychiatric Association in 1994. In the DSM-IV, tic disorders are classified into (1) transient tic disorders, (2) chronic motor or vocal tics, (3) Tourette's disorder, and (4) tic disorders not otherwise specified.

(1) Transient tic disorders have one or various motor and/or vocal tic symptoms, persisting from 4 weeks to less than 12 months. 95% of tic disorders fall into transient tic disorders, which disappear in a few months even without treatment. (2) Chronic motor or vocal tic disorders have one or various motor or vocal tic symptoms, persisting for 12 months or more, and tics do not disappear for 3 or more consecutive months. (3) Tourette's disorders have various motor and one or more vocal tic symptoms, persisting for 12 months or more, and tics do not disappear for 3 or more consecutive months. Tourette's disorders account for 0.2-0.5% of tic disorders, and one-third of them disappear entirely by late adolescence or early adulthood. One-third improves markedly with only mild symptoms such as blinking remaining. Symptoms persist in the remaining one-third. Others include (4) tic disorders not otherwise specified.

Psychogenic causes are involved in the etiologies of transient tic disorder and chronic motor or vocal tic disorder, and based on the pathophysiological studies and studies on Tourette's syndrome, functional and organic disorders of the central nervous system are also considered to underlie them.

The peptides in the present invention are compositions of 2 or more amino acids condensed by a peptide bond (also called amide bond); thus, those consisting of many amino acids are called polypeptides, and proteins are formed by 1 or more polypeptides. Hydrolysis of peptides produces original amino acids. Dipeptides are peptides formed via a peptide bond between 2 amino acids. The manufacturing methods of dipeptides include chemical synthesis, enzymatic synthesis, and proteolysis, although not particularly limited to them.

The dipeptides of the present invention include theanyl-theanine, theanyl-glutamine, glutaminyl-theanine, theanyl-alanine, alanyl-theanine, theanyl-asparagine, asparaginyl-theanine, theanyl-glutamic acid, glutamyl-theanine, theanyl-asparatic acid, aspartyl-theanine, theanyl-glycine, glycyl-theanine, theanyl-histidine, histidyl-theanine, theanyl-isoleucine, isoleucyl-theanine, theanyl-leucine, leucyl-theanine, theanyl-lysine, lysyl-theanine, theanyl-methionine, methionyl-theanine, theanyl-phenylalanine, phenylalanyl-theanine, theanyl-proline, prolyl-theanine, theanyl-serine, seryl-theanine, theanyl-threonine, threonyl-theanine, theanyl-tryptophan, tryptophanyl-theanine, theanyl-tyrosine, tyrosinyl-theanine, theanyl-valine, valyl-theanine, theanyl-arginine, arginyl-theanine, theanyl-cysteine, and cysteinyl-theanine.

The doses of theanine and dipeptides in the present invention to improve tic disorders are 0.1-500 mg/kg body weight, preferably 1-300 mg/kg body weight, and more preferably 2-100 mg/kg body weight. The doses of theanine and dipeptides can appropriately be determined according to the severity of tic disorders.

In addition, crude drugs, herbs, amino acids, vitamins, minerals, other foods and drinks, and materials and ingredients acceptable as pharmaceuticals can be used at the same time with the compositions in the present invention. Here, the crude drugs that are used include valerian, Angelicae radix, Paeoniae radix, tree peony, and Korean ginseng, although not particularly limited to them.

Herbs include anise, carrot seed, clove, coriander, cypress, cinnamon, juniper, ginger, sweet orange, pine needle, basil, patchouli, bitter orange, fennel, black pepper, bay, peppermint, bergamot, mandarin, myrrh, lemongrass, rosemary, grapefruit, cedar wood, citronella, sage, thyme, tea tree, violet leaf, vanilla, hyssop, eucalyptus, lime, lemon, ilang-ilang, cardamom, clary sage, jasmine, geranium, chamomile, Bulgarian rose, rose, olibanum, lavender, matricaria chamomilla, geranium, sandalwood nerori, verbena, petitgrain, vetiver, marjoram, Melissa officinalis, rosewood, Hypericum erectum, Hypericum perforatum, and kava kava.

The amino acids that are used include those that cross the blood-brain barrier. The amino acids transported via system L include long-chain neutral amino acids such as phenylalanine, tyrosine, leucine, isoleucine, valine, histidine, methionine, threonine, and theanine; the amino acids transported via system A include short-chain neutral amino acids such as alanine, glycine, serine, and threonine; the amino acids transported via system ASC include alanine, serine, and cysteine; the amino acids transported via other systems include taurine, glutamic acid, asparagine, lysine, and arginine.

The vitamins that are used include vitamin A, vitamin B₁, vitamin B₂, vitamin B₆, vitamin B₁₂, vitamin C, vitamin D, vitamin E, vitamin K, folic acid, niacin, lipoic acid, pantothenic acid, biotin, ubiquinone, and prostaglandin, including vitamin derivatives, although not limited to them.

The minerals that are used include calcium, iron, magnesium, copper, zinc, selenium, and potassium, although not limited to them.

As for the others, aloe, royal jelly, melatonin, placenta, propolis, isoflavone, soybean lecithin, egg-yolk lecithin, egg-yolk oil, chondroitin, cacao mass, collagen, vinegar, chlorella, spirulina, ginkgo biloba, green tea, du zhong tea, Royal Junshan Yinzhen, oolong tea, mulberry leaves, tencha, banaba, unsaturated fatty acid, sugars such as sugar alcohol and oligosaccharide, fungi such as bifidobacteria and red yeast rice, mushrooms such as Agaricus, Agaricus brazei murill and Reishi, fungi including Grifola frondosa, blueberry, prune, grape, olive, fruits including ume apricot and citrus, peanuts, almond, nuts and seeds such as sesame and pepper, green pepper, paprika, green onion, squash, cucurbitaceous fruit, carrot, burdock, mulukhiya, garlic, Japanese basil, wasabi, tomato, shallots, leaf vegetables, vegetables such as potatoes and beans, seaweeds such as brown seaweed, seafood, animal meat, poultry, whalemeat, and grains can be used; in addition, extracts, dry products, crude products, refined products, processed products, and brewed products of them can be used.

To consume the compositions of the present invention to improve tic disorders as an ingredient of foods and drinks or pharmaceuticals, they can take forms of solution, suspension, powder, and molded solid, containing the composition of the present invention, although not limited to them. More specifically, they include paste, soybean products, seasonings, mousse, jelly, frozen dessert, candy, chocolate, gum, cracker, cake, bread, soup, coffee, cocoa, teas such as green tea, oolong tea, black tea and herb tea, concentrated juice, from-concentrate juice, not from-concentrate juice, fruit-mixed juice, fruit juice with pulp, drinks with fruit juice, fruit/vegetable-mixed juice, vegetable juice, soda, soft drink, milky drink, dairy products, tablet, capsule, pharmaceuticals, and granule.

Formulations of the pharmaceuticals include internal medicine, injection drug, dermal patch, suppository, inhalation drug, and others. Internal medicines are ingested in the forms of conventional tablet, capsule, powder medicine, granulated medicine, drinkable preparation, and others. Injection drugs are injected into the body by intramuscular injection, intradermal injection, subcutaneous injection, intravenous injection, and others. Dermal patches are absorbed by the body after being mixed with conventional ointment. Suppositories are absorbed by the body after being mixed with conventional cacao butter, glycerogelatin, sodium stearate, propylene glycol monostearate, and others. Inhalation drugs are absorbed by the conventional methods; for instance, they are absorbed nasally or orally by the body by adding the invention to vapor or air.

The production methods of tablets in the present invention include direct tablet compression after mixing ingredients and tablet compression after wet or dry granulation of ingredients, although not limited to them. In addition, vehicles such as crystal cellulose and sugars can be mixed if needed. Furthermore, to improve tablet characteristics, lubricants such as sugar ester and polyglutamic acid ester can be used in combination. In general, corrigents used in foods, drinks, pharmaceuticals, and others can be used. Various flavors can be used as such corrigents, including lemon flavor, orange flavor, grapefruit flavor, chocolate flavor, dl-menthol, 1-menthol, and others.

### EXAMPLES

Hereinafter, the present invention is described in further detail with examples and test cases, although not limited to them.

### Example 1

### Production of Theanine by an Enzymatic Method

0.3 M glutamine and 1.5 M ethylamine hydrochloride in 0.05 M boric acid buffer (pH11) were reacted in the presence of 0.3 U glutaminase (commercial product) at 30° C. for 22 hours, and 225 nmol of L-theanine was obtained. Subsequently, the reaction solution was applied to Dowex 50×8 and Dowex 1×2 column chromatography (MUROMACHI CHEMICALS INC.), followed by ethanol treatment, to isolate the objective substance from the reaction solution.

The isolated substance was analyzed by an amino acid analyzer (Hitachi, Ltd.) and paper chromatography, and was confirmed to be L-theanine due to the same pattern of movement as that of the standard substance. Hydrolysis treatment with hydrochloric acid or glutaminase produced glutamic acid and ethylamine at a ratio of 1:1. Thus, the isolated substance was hydrolyzed by glutaminase, demonstrating the binding of ethylamine at the gamma position of glutamic acid. L-glutamic acid generated by hydrolysis was confirmed by glutamate dehydrogenase. Thus, 8.5 g of L-theanine was obtained.

### Example 2

### Extraction of Theanine from Tea Leaves

After extraction from 10 kg tea leaves (Camellia sinensis) with hot water, the extract was passed through a cation-exchange resin (Dowex HCR W-2; MUROMACHI CHEMICALS INC.) and eluted with 1N NaOH. The eluate was passed through activated charcoal (Taiko activated charcoal SG; Futamura Chemical Co., Ltd.), and was concentrated using a RO membrane (NTR729HF; Nitto Denko Corp.) with 15% ethanol, followed by purification with a column chromatography, and 24.8 g of L-theanine was produced by recrystallization.

L-theanine (trade name: Suntheanine; Taiyo Kagaku Co., Ltd.) was used in each test and production of each composition described below.

### Example 3

### Production of Tablets Combined with Theanine

For an example of tablets containing a composition combined with theanine to improve tic disorders, tablets combined with theanine were produced by mixing and compressing the materials shown in the following Table 1.

TABLE 1

| Ingredients | Mass (%) | Mass (g) |
|---|---|---|
| Frost sugar | 71.67 | 0.5375 |
| Trehalose | 10 | 0.075 |
| Theanine | 13.33 | 0.1 |
| Sucrose fatty acid ester | 1 | 0.0075 |
| Flavoring (lemon flavor) | 4 | 0.03 |
| Total | 100 | 0.75 |

Namely, each material was mixed according to the above composition, and after granulation, tablets were compressed to 0.75 g/tablet.

### Example 4

### Production of Theanyl-Theanine

0.32 M L-theanine in 0.05 M boric acid buffer (pH11) was reacted in the presence of 0.3 U glutaminase (commercial product) at 30° C. for 22 hours, and 150 nmol of theanyl-theanine was obtained. Subsequently, the reaction solution was applied to Dowex 50×8 and Dowex 1×2 column chromatography (MUROMACHI CHEMICALS INC.) to isolate the objective substance from the reaction solution.

### Example 5

### Production of Tablets Combined with Theanyl-Theanine

For an example of tablets containing a composition combined with theanyl-theanine to improve tic disorders, tablets combined with theanyl-theanine were produced by mixing and compressing the materials shown in the following Table 2.

TABLE 2

| Ingredients | Mass (%) | Mass (g) |
|---|---|---|
| Frost sugar | 71.67 | 0.5375 |
| Trehalose | 10 | 0.075 |
| Theanyl-theanine in example 4 | 13.33 | 0.1 |
| Sucrose fatty acid ester | 1 | 0.0075 |
| Flavoring (lemon flavor) | 4 | 0.03 |
| Total | 100 | 0.75 |

Namely, each material was mixed according to the above composition, and after granulation, tablets were compressed to 0.75 g/tablet.

### Example 6

### Production of a Mixture of Theanyl-Glutamine and Glutaminyl-Theanine

0.4 M L-theanine and 0.4 M L-glutamine in 0.05 M boric acid buffer (ph11) were reacted in the presence of 0.3 U glutaminase (commercial product) at 30° C. for 22 hours to obtain 120 nmol of a mixture of theanyl-glutamine and glutaminyl-theanine. Subsequently, the reaction solution was applied to Dowex 50×8 and Dowex 1×2 column chromatography (MUROMACHI CHEMICALS INC.) to isolate the objective substance from the reaction solution. Structural analysis was conducted on the theanyl-glutamine and glutaminyl-theanine by mass spectrum analysis and NMR for confirmation.

### Example 7

### Production of Tablets Combined with a Mixture of Theanyl-Glutamine and Glutaminyl-Theanine

For an example of tablets containing a composition combined with a mixture of theanyl-glutamine and glutaminyl-theanine to improve tic disorders, tablets combined with theanyl-glutamine and glutaminyl-theanine were produced by mixing and compressing the materials shown in the following Table 3.

TABLE 3

| Ingredients | Mass (%) | Mass (g) |
|---|---|---|
| Frost sugar | 71.67 | 0.5375 |
| Trehalose | 10 | 0.075 |
| A mixture of theanyl-glutamine and | 13.33 | 0.1 |
| glutaminyl-theanine in example 6 | | |
| Sucrose fatty acid ester | 1 | 0.0075 |
| Flavoring (lemon flavor) | 4 | 0.03 |
| Total | 100 | 0.75 |

Namely, each material was mixed according to the above composition, and after granulation, tablets were compressed to 0.75 g/tablet.

<Test Case 1> A Case with Improved Tic Disorder

Patient: 12 years old (Sixth grade male elementary school student);

Body weight: 37 kg

Chief complaint: shaking hands and head

Family history: four family members including a one year younger brother and parents; no special note

Previous history: bronchial asthma in early childhood

History: Since being elected as temporary class representative of the fourth grade class of elementary school, the patient had blinking eyes and subsequently shook his face and shoulders; thus, he visited a nearby hospital and received diazepam. Since there was no effect, the patient was referred to a department of paediatric neurology to receive pimozide and got relief in 3 months. In the following year, around the time of moving to Osaka, the patient suffered a relapse of symptoms of shaking his face and shoulders again and thus visited the pediatric department of O hospital and received haloperidol. However, the patient has continued to receive an increased dosage until now due to an insufficient effect, but the adverse effects of haloperidol (trade name: Serenase (trademark); Dainippon Pharmaceutical Co. Ltd.) such as a speech defect appeared recently; thus, an antiparkinson drug had also been given.

This time, since the course was unsatisfactory, the patient received 10 tablets combined with theanine in example 3 (1 g theanine) daily for 20 days; the symptoms of tic disorders have completely disappeared. Meanwhile, there were no adverse effects specially noted for theanine.

<Test Case 2> A Case with Improved Tourette's Disorder

Patient: 13 years old; male child; body weight: 43 kg

Chief complaint: tic

History: The patient had started blinking since around 5 years of age, and subsequently jumped and made voice sounds like “oof oof.” Since 11 years of age (fifth grade), the patient had showed additional tic symptoms such as licking his lips and thrusting out his tongue; about 3-month cycles of waxing and waning were repeated. The tics were milder when he was in school or with others, and were more frequently observed in the presence of only his family. At 12 years of age (in the third semester of the sixth grade), he visited our clinic worrying about bullying at his junior high school. On the first visit, besides the above-mentioned symptoms, the patient continuously showed symptoms such as esotropia-like adducted eyes, convulsive grimace of the nose, cheeks, and mouth, swollen nose, and coughing. In addition, the patient continuously moved his hands and legs and tended to be restlessly absent-mindedly hyperactive.

Family history: four family members including parents and younger brother (third grade)

Psychosocial case history: The patient was always crying due to inherent sensitivity; thus, his mother felt anxious about child-rearing. He was severely afraid of strangers and timid, and often cried for fear. He had a poor school record. He often cut into others' conversation and was too talkative. His mother can get along with his younger brother casually, but is feeling awkwardness and pain with the patient due to the lack of closeness. His father is not nagging so much, and seems to accept the patient. Clinical course: The patient was diagnosed as having Tourette's disorder, and the disease was explained to his parents. To alleviate the symptoms, haloperidol (trade name: Serenase (trademark); Dainippon Pharmaceutical Co. Ltd.) was used. The dosage was gradually increased from 0.25 mg to 0.5 mg and thereby alleviated the symptoms such as tics and restlessness; thus, the administration has been continued. However, drowsiness was caused as an adverse effect, which caused nodding during study with resulting negative effects on his study. Instead of serenase, 15 tablets combined with theanine in example 3 (1.5 g theanine) were given daily for 45 days and thereby completely improved his tic disorders. Meanwhile, there were no adverse effects specified for theanine.

The administration of theanine is scheduled to be continued until the end of puberty. The patient has joined his junior high school track club, and has tics, although unnoticeable. The patient has become a bit disobedient and often falls silent, but is highly motivated with study.

<Test Case 3> A Case with Improved Tic Disorder

Patient: 7-year-old male child; height 121 cm; body weight 20 kg

Chief complaint: repetitive guttural

History: As for the history, at around the time when his father was acutely hospitalized due to renal calculi in the summer of 1989, the patient often showed blinking and gestures of pulling clothes. He was diagnosed as having tics.

The mother disciplined the child by scolding. She was pressed with child-rearing and housework and had little free time; thus, she was always nagging due to frustration. In July 1990, the patient developed involuntary movements of shaking his shoulders up and down, and in addition, repetitive guttural. In Sep. 6, 1990, the patient first visited the clinic. The affected child was thin with abdominal palpitation and spasms of the musculus rectus abdominis. Since October, the patient received 8 tablets combined with theanyl-theanine in example 5 (0.8 g theanyl-theanine) daily for 30 days. The tic symptoms of the patient almost disappeared with the use of the tablets combined with theanyl-theanine in example 5. In addition, there were no adverse effects specified for theanyl-theanine.

<Test Case 4> A Case with Improved Tic Disorder

Patient: 5 years old

Chief complaint: Blinking

History: The patient was crying out and thus was forced to plunge into a pool against his will. Since that night, the patient had developed blinking tics. The patient was diagnosed as having tics. The patient received 5 tablets combined with a mixture of theanyl-glutamine and glutaminyl-theanine (0.5 g as a mixture of theanyl-glutamine and glutaminyl-theanine) in example 7 daily for 20 days. By taking these tablets, the tic symptoms of the patient have almost disappeared. Meanwhile, there were no adverse effects specified for the mixture of theanyl-glutamine and glutaminyl-theanine.

### Example 8

### Production of Candies Combined with Theanine

For an example of foods and drinks or pharmaceuticals containing a composition combined with theanine to improve tic disorders, candies combined with theanine were produced using the materials shown in the following table 4.

TABLE 4

| Ingredients | Mass (kg) |
|---|---|
| Granulated sugar | 64 |
| Starch syrup | 23 |
| Theanine | 10 |
| Flavoring (lemon flavor) | 0.05 |
| 50% tartaric acid | 1 |
| Water | 30 |

64 kg granulated sugar was dissolved in 20 kg water by heating to 110° C., and after adding the remaining 10 kg water, in which theanine was dissolved, and starch syrup, the temperature was increased to 145° C. After turning off the heat, 50% tartaric acid was added and mixed. After being cooled to 75-80° C., the mixture was shaped with a molding roller to produce candies combined with theanine.

As a result of measurement of theanine in the candies, the content was 89.6 mg/g (1.2 g theanine/candy).

### Example 9

### Production of Blueberry Drink Combined with Theanine

For an example of foods and drinks or pharmaceuticals containing a composition combined with theanine to improve tic disorders, a drink combined with theanine was produced using the materials shown in the following table 5.

TABLE 5

| Ingredients | Mass (kg) |
|---|---|
| Fructose-glucose | 12 |
| Concentrated blueberry juice | 1 |
| ⅕ clear lemon juice | 0.4 |
| Citric acid Na | 0.05 |
| 50% citric acid Na (crystal) | For pH adjustment |
| Theanine | 0.1 |
| Flavoring (blueberry flavor) | 0.05 |
| Water | Moderate amounts |
| Total amount | 100 |

Fructose-glucose, concentrated blueberry juice, 1/5 clear lemon juice, citric acid Na, and theanine were added to water and dissolved by shaking. The solution was adjusted to pH 3.1 using 50% citric acid Na (crystal) and heated to 95° C., then after adding flavorings, 100 mL each was filled into a container to produce blueberry drink combined with theanine. As a result of measurement of theanine in the blueberry juice, the content was 98.3 mg/100 mL.

### Example 10

### Production of Grapefruit Drink Combined with Theanyl-Theanine

For an example of foods and drinks or pharmaceuticals containing a composition combined with theanyl-theanine to improve tic disorders, a drink combined with theanyl-theanine was produced using the materials shown in the following table 6.

TABLE 6

| Ingredients | Mass (kg) |
|---|---|
| Fructose-glucose | 6 |
| Theanyl-theanine in example 4 | 0.1 |
| Ferric pyrophosphate | 0.06 |
| Placenta extract | 0.01 |
| 100% grapefruit juice | 30 |
| Citric acid Na | For pH adjustment |
| Flavoring (grapefruit flavor) | 0.05 |
| Water | Moderate amounts |
| Total amount | 100 |

Fructose-glucose, theanyl-theanine, ferric pyrophosphate, placenta extract, and 100% grapefruit juice were added to water and dissolved by shaking. The solution was adjusted to pH 3.1 using citric acid Na and heated to 95° C., and after adding flavorings, 100 mL each was filled into a container to produce a grapefruit drink combined with theanyl-theanine. As a result of measurement of theanyl-theanine in the grapefruit juice, the content was 96.4 mg/100 mL.

Examples of the present invention are shown as follows:

(1) A composition to improve tic disorders containing theanyl-theanine as an active ingredient

(2) A composition to improve tic disorders containing theanyl-glutamine as an active ingredient

(3) A composition to improve tic disorders containing glutaminyl-theanine as an active ingredient

(4) A composition to improve tic disorders containing theanyl-alanine as an active ingredient

(5) A composition to improve tic disorders containing alanyl-theanine as an active ingredient

(6) A composition to improve tic disorders containing theanyl-asparagine as an active ingredient

(7) A composition to improve tic disorders containing asparaginyl-theanine as an active ingredient

(8) A composition to improve tic disorders containing theanyl-glutamic acid as an active ingredient

(9) A composition to improve tic disorders containing glutamyl-theanine as an active ingredient

(10) A composition to improve tic disorders containing theanyl-glycine as an active ingredient

(11) A composition to improve tic disorders containing glycyl-theanine as an active ingredient

(12) A composition to improve tic disorders containing theanyl-histidine as an active ingredient

(13) A composition to improve tic disorders containing histidyl-theanine as an active ingredient

(14) A composition to improve tic disorders containing theanyl-isoleucine as an active ingredient

(15) A composition to improve tic disorders containing isoleucyl-theanine as an active ingredient

(16) A composition to improve tic disorders containing theanyl-leucine as an active ingredient

(17) A composition to improve tic disorders containing leucynyl-theanine as an active ingredient

(18) A composition to improve tic disorders containing theanyl-lysine as an active ingredient

(19) A composition to improve tic disorders containing lysyl-theanine as an active ingredient

(20) A composition to improve tic disorders containing theanyl-methionine as an active ingredient

(21) A composition to improve tic disorders containing methionyl-theanine as an active ingredient

(22) A composition to improve tic disorders containing theanyl-phenylalanine as an active ingredient

(23) A composition to improve tic disorders containing phenylalanyl-theanine as an active ingredient

(24) A composition to improve tic disorders containing theanyl-proline as an active ingredient

(25) A composition to improve tic disorders containing prolyl-theanine as an active ingredient

(26) A composition to improve tic disorders containing theanyl-serine as an active ingredient

(27) A composition to improve tic disorders containing seryl-theanine as an active ingredient

(28) A composition to improve tic disorders containing theanyl-threonine as an active ingredient

(29) A composition to improve tic disorders containing threonyl-theanine as an active ingredient

(30) A composition to improve tic disorders containing theanyl-tryptophan as an active ingredient

(31) A composition to improve tic disorders containing tryptophanyl-theanine as an active ingredient

(32) A composition to improve tic disorders containing theanyl-tyrosine as an active ingredient

(33) A composition to improve tic disorders containing tyrosinyl-theanine as an active ingredient

(34) A composition to improve tic disorders containing theanyl-valine as an active ingredient

(35) A composition to improve tic disorders containing valyl-theanine as an active ingredient

(36) A composition to improve tic disorders containing theanyl-arginine as an active ingredient

(37) A composition to improve tic disorders containing arginyl-theanine as an active ingredient

(38) A composition to improve tic disorders containing theanyl-cysteine as an active ingredient

(39) A composition to improve tic disorders containing cysteinyl-theanine as an active ingredient

(40) A composition to improve tic disorders containing aspartyl-theanine as an active ingredient

(41) A composition to improve tic disorders containing theanyl-asparatic acid as an active ingredient

(42) A composition to improve tic disorders containing glutaminyl-alanine as an active ingredient

(43) A composition to improve tic disorders containing alanyl-glutamine as an active ingredient

(44) A composition to improve tic disorders containing glutaminyl-asparagine as an active ingredient

(45) A composition to improve tic disorders containing asparaginyl-glutamine as an active ingredient

(46) A composition to improve tic disorders containing glutaminyl-glutamic acid as an active ingredient

(47) A composition to improve tic disorders containing glutaminyl-glycine as an active ingredient

(48) A composition to improve tic disorders containing glycyl-glutamine as an active ingredient

(49) A composition to improve tic disorders containing glutaminyl-histidine as an active ingredient

(50) A composition to improve tic disorders containing histidyl-glutamine as an active ingredient

(51) A composition to improve tic disorders containing glutaminyl-isoleucine as an active ingredient

(52) A composition to improve tic disorders containing isoleucyl-glutamine as an active ingredient

(53) A composition to improve tic disorders containing glutaminyl-leucine as an active ingredient

(54) A composition to improve tic disorders containing leucyl-glutamine as an active ingredient

(55) A composition to improve tic disorders containing glutaminyl-lysine as an active ingredient

(56) A composition to improve tic disorders containing lysyl-glutamine as an active ingredient

(57) A composition to improve tic disorders containing glutaminyl-methionine as an active ingredient

(58) A composition to improve tic disorders containing methionyl-glutamine as an active ingredient

(59) A composition to improve tic disorders containing glutaminyl-phenylalanine as an active ingredient

(60) A composition to improve tic disorders containing phenylalanyl-glutamine as an active ingredient

(61) A composition to improve tic disorders containing glutaminyl-proline as an active ingredient

(62) A composition to improve tic disorders containing prolyl-glutamine as an active ingredient

(63) A composition to improve tic disorders containing glutaminyl-serine as an active ingredient

(64) A composition to improve tic disorders containing seryl-glutamine as an active ingredient

(65) A composition to improve tic disorders containing glutaminyl-threonine as an active ingredient

(66) A composition to improve tic disorders containing threonyl-glutamine as an active ingredient

(67) A composition to improve tic disorders containing glutaminyl-tryptophan as an active ingredient

(68) A composition to improve tic disorders containing tryptophanyl-glutamine as an active ingredient

(69) A composition to improve tic disorders containing glutaminyl-tyrosine as an active ingredient

(70) A composition to improve tic disorders containing tyrosyl-glutamine as an active ingredient

(71) A composition to improve tic disorders containing glutaminyl-valine as an active ingredient

(72) A composition to improve tic disorders containing valyl-glutamine as an active ingredient

(73) A composition to improve tic disorders containing glutaminyl-arginine as an active ingredient

(74) A composition to improve tic disorders containing arginyl-glutamine as an active ingredient

(75) A composition to improve tic disorders containing glutaminyl-cysteine as an active ingredient

(76) A composition to improve tic disorders containing cysteinyl-glutamine as an active ingredient

(77) A composition to improve tic disorders containing glutaminyl-asparagine as an active ingredient

(78) A composition to improve tic disorders containing asparaginyl-glutamine as an active ingredient

(79) A composition to improve tic disorders containing glutaminyl-asparatic acid as an active ingredient

(80) A composition to improve tic disorders containing aspartyl-glutamine as an active ingredient

(81) A composition to improve tic disorders containing glutaminyl-glutamine as an active ingredient

What is claimed is:

1. Compositions to improve tic disorders, which contain an amino acid that crosses the blood-brain barrier and/or a peptide with an amino acid that crosses the blood-brain barrier as an active ingredient.

2. Compositions to improve tic disorders, wherein the amino acid according to claim 1 is theanine.

3. Compositions to improve tic disorders, wherein the peptide according to claim 1 is a dipeptide consisting of (A) theanine and (B) one of the amino acids selected from theanine, glutamine, alanine, asparagine, asparatic acid, glutamic acid, glycine, histidine, isoleucine, leucine, lysine, methionine, phenylalanine, proline, serine, threonine, tryptophan, tyrosine, valine, arginine, and cysteine.

4. Foods, drinks, and pharmaceuticals containing the composition described in claim 1.

5. Foods, drinks, and pharmaceuticals containing the composition described in claim 2.

6. Foods, drinks, and pharmaceuticals containing the composition described in claim 3.

7. Production methods of tablets containing the composition described in claim 1.

8. Production methods of tablets containing the composition described in claim 2.

9. Production methods of tablets containing the composition described in claim 3.

10. Production methods of tablets containing the composition described in claim 4.
